(19) United States
(12) Patent Application Publication
(10) Pub. No.: US 2022/0288360 A1
(43) Pub. Date: Sep. 15, 2022

(54) AUTOMATED SYSTEM AND METHOD FOR AIDING IN LYMPHATIC DRAINAGE

(71) Applicants: India Reid Reynolds, Winterville, NC (US); Darryl Verdale Reynolds, Winterville, NC (US)

(72) Inventors: India Reid Reynolds, Winterville, NC (US); Darryl Verdale Reynolds, Winterville, NC (US)

(21) Appl. No.: 17/198,671

(22) Filed: Mar. 11, 2021

(51) Int. Cl. A61M 27/00

(52) U.S. Cl. CPC A61M 27/00; A61M 2205/3553; A61M 2209/088; A61M 2202/0405

(57) ABSTRACT

An automated system and method, wherein a movement device aids in lymphatic drainage and activation of the lymphatic system by applying a skin safe medium on the skin or on or near the lymph gland(s) creating an automated repetitive drainage effect that may allow patients to be more successful at reducing and managing their swelling.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a device for aiding in lymphatic drainage. In particular, it relates to a movement device for stretching and relaxing the skin near the lymphatic system which aids in lymphatic drainage and increased activity of the lymphatic system.

### Description of Related Art

The lymphatic system, among other things, assists in removing toxins from the body and is part of the body's immune system that helps fight infection. It also has a significant role in the circulatory system as it enables the interstitial proteins that cannot be reabsorbed back into the venous system to return to central circulation from all areas of the body. When there is impairment to this function, regardless as to primary or secondary causes, these proteins and the fluid that they are solubilized in begin to accumulate and cause swelling of the area. This can lead to a disease state called lymphedema.

Historically, many have considered lymphedema to be just a cosmetic problem. Unfortunately, the problems that result from lymphedema are far reaching and may involve the inability to find proper clothing, result in the loss of mobility/function of the musculoskeletal system, cause suffering from debilitating pain, and increases an individual's susceptibility to various infections (e.g., cellulitis) that can be fatal if not treated in a timely manner. It may also affect patients psychologically (e.g., depression) and affect their relationships with loved ones that assume a caregiver role that is laborious and needed for the lifetime of the individual.

The gold standard for lymphedema therapy is Complete Decongestive Therapy (CDT). Complete Decongestive Therapy is customized to address the unique needs of each individual. This is necessary because the stages of lymphedema and clinical presentations vary and comorbidities of the individual must be considered. In general, CDT incorporates manual lymphatic drainage (MLD), compression therapy, exercise, and skin care. Complete Decongestive Therapy has two phases, the intensive phase and the maintenance phase. During the intensive phase, the certified lymphedema therapist incorporates various advanced skills to customize the program to the needs of the patient in order to achieve decongestion of the involved area. The training of the patient and/or their caregiver allows for transition to the maintenance phase. This phase involves less complex skills, but still incorporates the four modalities of CDT. Additional methods to assist with removal of excess fluid include Kinesio® taping and pneumatic compression.

Lymphedema is currently incurable, but can be managed successfully. Long-term success of maintaining decongestion is directly correlated with ongoing, lifetime compliance with daily use of the aforementioned strategies in various degrees. This is challenging for many people. Manual Lymphatic Drainage can be hard to master, as the proper hand motions, pressure applied, and sequence of performance is critical in order to be successful. For some people, areas of the body that require treatment may not be accessible (e.g., the back) and will require someone else to perform part or all of the treatment. A suitable caregiver may be unavailable or unwilling to provide this lifetime treatment, however, application of pneumatic compression pumps requires little skill. Unfortunately, as is also the case with MLD, to maintain the drainage achieved with compression pumps, the involved area must be donned with compression bandaging or garments. Many patients find the garments uncomfortable, limiting in mobility, and/or cosmetically unappealing. Hence, long term success of achieving and maintaining decongestion of the involved area eludes many. There is a great need to have a means for lymphatic drainage that overcomes one or more of the above problems that occur using manual systems. The device described below can be used to treat lymphedema, as well as any other disease states (e.g., post-operative swelling, edema associated with wounds, venous insufficiency, etc.).

### BRIEF SUMMARY OF THE INVENTION

The present invention relates to an automated system and method for stimulating lymphatic drainage by attachment of a medium to the skin, wherein a wire associated with the medium is automatically retracted and extended, as desired. This creates a draining effect which mimics the well-documented basic drainage effect of MLD on the initial lymphatic vessels.

Accordingly, in one embodiment, there is an automated system for aiding in lymphatic drainage by use of a system on the skin, on or near the lymph glands, at least a portion of the automated system, the system comprising:
a) a skin safe medium designed to attach to skin, the skin safe medium having a wire positioned on one of the outside or inside of the skin safe medium;
b) a movement device having a first end of the wire attached to it, wherein the movement device retracts and extends the wire in a manner that moves the skin safe medium and causes stretching and relaxing of the skin; and
c) a wireless remote control for sending a signal to a receiver connected to the movement device that controls the functioning of the movement device for retracting and extending the wire.

In another embodiment, there is a method for aiding in lymphatic drainage by use of a system on the skin, on or near the lymph glands, of at least a portion of an automated system, the system comprising:
a) a skin safe medium having a wire positioned on one of the outside or inside of the skin safe medium;
b) a movement device having a first end of the wire attached to it, wherein the movement device retracts and extends the wire in a manner that moves the skin safe medium and causes stretching and relaxing of the skin;
c) a wireless remote control for sending a signal to a receiver connected to the movement device that controls the functioning of the movement device for retracting and extending the wire; and
d) wherein the system is activated causing the retraction and extension of the wire, creating a drainage effect on the skin and on any underlying lymph vessels.

### BRIEF DESCRIPTION OF THE DRAWINGS

FIG. 1 is a perspective view of the system of the present invention deployed on an individual for draining a lymph gland.

### DETAILED DESCRIPTION OF THE INVENTION

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

As used herein, the term “automated system” refers to the system doing the work instead of a manual approach (i.e., Manual Lymphatic Drainage “MLD”) for aiding in lymphatic drainage. The system, once placed on the patient, needs only to be engaged (e.g., on/off wireless engagement) since the system does the work (i.e., automated massage system). However, this does not negate the additional drainage the patient may benefit from if pre-treatment MLD techniques are provided (e.g., softening of fibrotic tissues, direct drainage of lymph nodes associated with the treatment pathway).

As used herein, the term “aiding in lymphatic drainage” refers to the well-known principle of stretching of the skin in the proper direction and sequence to aid in lymphatic drainage. This well-known principle is applied in MLD. However, the amount of pressure, the hand motions, the direction of flow, and time vary so greatly that the effects of MLD are difficult for patients to duplicate repetitively, which is part of the goal of CDT. The present invention is able to create a basic motion performed by the hands when performing MLD, but does it automatically with a wireless remote control and a skin safe medium activated by a movement device.

As used herein, the term “aiding in” refers to adding assistance to improve, encourage, increase, and the like in lymphatic drainage.

As used herein, the term “skin safe medium” refers to a material capable of adhering to or to be attached to (e.g., adhesives such as glues, and the like) the skin in the area of skin surrounding the lymph gland. In one embodiment, it can be attached to skin for a period of at least seven days. It also must have the capability of a wire being imbedded in or placed on the skin safe medium sufficient enough that pulling on the wire causes the skin safe material to stretch or pull on the skin where it is attached. The skin safe medium includes, for example, polymers, a hydrocolloid, and silicone.

As used herein, the term “attach to skin” refers to the ability of the skin safe medium to attach to the skin for at least about seven days. Attachment by adhesives that are compatible with the skin safe medium and the patient's skin are contemplated. Skin adhesives are well known in the art.

As used herein, the term “wire” refers to a metal in the form of a usually very flexible thread or slender rod. The wire attaches to the skin safe medium material at a distal end and to a movement device at a proximal end.

As used herein, the term “movement device” refers to a remote controlled motor and the like, that attaches to the wire at a proximal end and can pull on the wire, then return to its original position, thus causing stretching of the skin attached to the skin safe medium material. Because treatment must be customized to the individual's clinical presentation of lymphedema, the exact parameters of treatment frequency, hold time of stretching, intensity of stretch, etc., will need to be set by the treating lymphedema specialist.

As used herein, the term “wire attached” refers to, as noted above, a distal end attached to the skin safe medium and a proximal end attached to the movement device. In one embodiment, the wire is attached to the skin safe medium.

As used herein, the term “motor that retracts” refers to a part of the movement device that a wire can attach to and repetitively pull on the proximal end of the wire, then allow it to return to its original position. The motor has a wireless receiver and operating the motor is controlled by a device having a transmitter that delivers a message to the motor (e.g., a smart phone or smart phone app). In an example embodiment, a smart phone has an app that can turn the motor on and off. The app will also include features that will allow treatment intensity, duration, and frequency to be set. An activity log of the motor activity will also be available through the app.

In the use of the present invention, a skin safe medium is attached to the skin to direct flow of drainage toward operating lymph nodes, the skin safe medium having a wire in or on the skin safe medium. A wireless remote control, like a cell phone app, sends a signal to a movement device, which is attached to the proximal end of the wire causing retraction and extension of the wire, which creates a drainage effect on any underlying lymph vessels.

### DRAWINGS

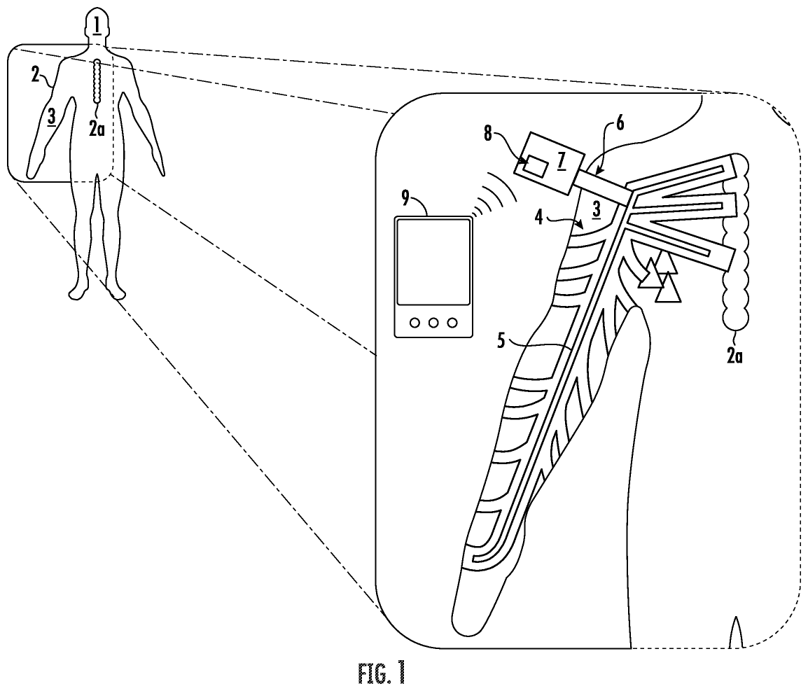

Now referring to the drawings, FIG. 1 is a perspective view of the system of the present invention for aiding in lymphatic drainage. In this view, patient 1 has a swollen left arm 2 and needs drainage directed to the lymph nodes 2a. A skin safe medium 4 is attached to skin 3. The skin safe medium 4 has a wire 5 attached on the outside or placed into the skin safe medium. The proximal end of the wire 6 is attached to a motor 7 (movement device) for pull of the wire. The motor 7 has an internal wireless receiver 8 for receiving commands for turning on or off, regulating time, regulating speed, and the like. Instructions are sent from smartphone 9 to the internal wireless receiver 8.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1. An automated system for aiding in lymphatic drainage by use of a system on the skin, on or near the lymph glands, at least a portion of the automated system, the system comprising:
a) a skin safe medium designed to attach to skin, the skin safe medium having a wire positioned on one of the outside or inside of the skin safe medium;
b) a movement device having a first end of the wire attached to it, wherein the movement device retracts and extends the wire in a manner that moves the skin safe medium and causes stretching and relaxing of the skin; and
c) a wireless remote control for sending a signal to a receiver connected to the movement device that controls the functioning of the movement device for retracting and extending the wire.

2. The automated system for aiding in lymphatic drainage according to claim 1 wherein the skin safe medium is selected from the group consisting of a polymer, a hydrocolloid, and silicone.

3. The automated system for aiding in lymphatic drainage according to claim 1 wherein the skin safe medium is attached to skin by an adhesive.

4. The automated system for aiding in lymphatic drainage according to claim 1 wherein the remote control controls at least one of frequency of activation, time of activation, and the degree of pull on the wire by the movement device.

5. The automated system for aiding in lymphatic drainage according to claim 1 wherein the wireless remote control utilizes at least one of radio transmission, Bluetooth®, Z-Wave®, and WiFi for sending a signal.

6. The automated system for aiding in lymphatic drainage according to claim 1 wherein the movement device is a motor.

7. A method for aiding in lymphatic drainage by use of a system on the skin, on or near the lymph glands, at least a portion of an automated system, the system comprising:
a) a skin safe medium having a wire positioned on one of the outside or inside of the skin safe medium;
b) a movement device having a first end of the wire attached to it, wherein the movement device retracts and extends the wire in a manner that moves the skin safe medium and causes stretching and relaxing of the skin;
c) a wireless remote control for sending a signal to a receiver connected to the movement device that controls the functioning of the movement device for retracting and extending the wire; and
d) wherein the system is activated causing the retraction and extension of the wire, creating a drainage effect on the skin and on any underlying lymph vessels.
